(19) United States
(12) Patent Application Publication
(10) Pub. No.: US 2022/0062910 A1
(43) Pub. Date: Mar. 3, 2022

(54) POLYMERASE CHAIN REACTION SYSTEM

(71) Applicant: Robert Hering, Clermont, FL (US)

(72) Inventor: Robert Hering, Clermont, FL (US)

(21) Appl. No.: 17/463,657

(22) Filed: Sep. 1, 2021

Related U.S. Application Data
(60) Provisional application No. 63/073,650, filed on Sep. 2, 2020.

(51) Int. Cl. B01L 7/00; B01L 3/00; C12Q 1/686

(52) U.S. Cl. CPC B01L 7/52; B01L 3/508; C12Q 1/686; B01L 2200/025; B01L 2200/10; B01L 2200/16; B01L 2300/1811; B01L 2300/025; B01L 2300/0609; B01L 2300/0654; B01L 2300/0663; B01L 2300/12; B01L 2300/1894; B01L 2300/023

(57) ABSTRACT

A Polymerase Chain Reaction (PCR) device used to detect infectious agents and certain diseases. The device provides a quantitative PCR that amplifies the target DNA sequence, while monitoring the amplitude of signal originating from DNA-intercalating probes, typically fluorescence, to quantify the amount of DNA being amplified. The device is used in combination with other medical devices to obtain the user's physical exam data. A combination of the PCR machine and above-mentioned other instruments with a network communication device (TOTUS) sends the physical exam and PCR information to servers, and/or physicians, pharmacists and other medical professionals to provide AI decisions.

### PRIORITY CLAIM

In accordance with 37 C.F.R. 1.76, a claim of priority is included in an Application Data Sheet filed concurrently herewith. Accordingly, the present invention claims priority to U.S. Provisional Patent Application No. 63/073,650, entitled “POLYMERASE CHAIN REACTION SYSTEM”, filed Sep. 2, 2020; the contents of which are incorporated herein by reference.

### FIELD OF THE INVENTION

This invention is directed to the field of human diagnostics and, in particular, to a polymerase chain reaction system using a PCR device to detect infectious agents and cancers incorporated into a system for recording and distribution using the PCR device as a diagnostic tool.

### BACKGROUND OF THE INVENTION

It is well known that early diagnosis and prompt treatment of an individual's health issue provides the best opportunity to reduce the risk of spreading to other individuals if the health issue is contagious, and addressing the needs of the individual to restore them to good health.

Depending on the health issue or disease, numerous devices have been employed with the goal of quick diagnostics. Many devices have been introduced as rapid diagnostics tests (RDT) so as to reinforce the need for immediate testing to complement early diagnosis and prompt treatment. Such devices preferably have low manufacturing costs and can provide a first level of screening to determine the necessity of follow-up tests. Devices can vary based upon the test employed; antigen-based devices require a high-level of pathogens in the patient sample compared to a Polymerase Chain Reaction (PCR) which can be detected as a positive result.

According to the Centers for Disease Control (CDC) and World Health Organization (WHO), the use of a PCR is recommended because a PCR reveals both the presence and the quantity of the pathogen and have high sensitivity and specificity, wherein diseases such as Malaria and HIV can be promptly detected. A PCR can be designed to amplify a specific sequence of DNA, providing high specificity in diagnostics.

Complications resulting from delayed diagnosis include irreversible organ damage, increased usage of medical supplies to contain exacerbated symptoms, and failure to quarantine early. For instance, P. falciparum can cause ischemic insults to the brain, lungs and kidneys when not treated promptly, which frequently can cause mortality to children and irreversible damage even if patients recover from a disease.

When patients enter the advanced clinical phase from infectious diseases, hospitalization with intravenous support/patient monitoring is required. In addition, an antibiotic/antiparasitic is frequently required to deal with systemic inflammation caused by an infectious agent.

Containment for HIV at a community level is always challenged by frequent quarantine failures due to undiagnosed/misdiagnosed asymptomatic carriers. All of which necessitates the need for early diagnosis by use of a PCR device.

Attempts to lower the price of a PCR include the use of a heater and temperature sensor integrated into a handheld device to perform quantitative PCR testing. Another attempt was a pocket-sized PCR thermocycler designed by fabricating heating blocks with various temperatures for convective-flow of PCR samples. Most devices developed under this motivation had custom parts that needed to be manufactured with unique or expensive processes, such as printed circuit boards (PCB), microfluidics, and micro-fabricated devices, in order to miniaturize the size of the device for portability.

However, the high cost for a conventional PCR device, due to complexity of instrumentation and maintenance, leaves the PCR device seldom used when immediate results are required.

What is needed in the industry is a RDT based on a low cost PCR device to increase the accessibility of diagnostic testing.

### SUMMARY OF THE INVENTION

Disclosed is a Polymerase Chain Reaction (PCR) device used to detect infectious agents, such as virus, Mycoplasma, Rickettsia and bacteria in the users and their environment. The PCR device can also be used to detect diseases such as cancer and Alzheimer's.

An objective of the invention is to provide a low cost PCR device capable of providing a conclusive result in determining of an infection, strain or type of pathogen, and level of infection.

Still another objective of the invention is to provide a quantitative PCR that amplifies the target DNA sequence, while monitoring the amplitude of signal originating from DNA-intercalating probes, typically fluorescence, to quantify the amount of DNA being amplified. The characteristic of the fluorescence signal reveals the original quantity in the sample before amplification.

Yet another objective of the invention is to provide low-cost, easy to manufacture and low maintenance PCR equipment for use at a primary doctor level, wherein the PCR device enables rapid on-site diagnosis which brings a great benefit to community health. Treatments can be given before infection causes more damage to patients. The clinic can be more efficient on distributing their limited medical resources, and authorities can set up quarantine for rapid containment of infectious disease if needed.

Yet still another objective of the invention is to provide a method and device that, by uploading and sharing the design files online, a low-cost qPCR device may provide easier access to a robust diagnosis protocol for various infectious diseases, such as HIV and malaria, wherein a disposable one-step PCR sampling device can use a one-step PCR reagent kit to perform assays.

Other objectives and advantages of this invention will become apparent from the following description taken in conjunction with the accompanying drawings wherein are set forth, by way of illustration and example, certain embodiments of this invention. The drawings constitute a part of this specification, include exemplary embodiments of the present invention, and illustrate various objects and features thereof.

### BRIEF DESCRIPTION OF THE FIGURES

FIG. 1 is a pictorial view of a qPCR aluminum heating element of the device;

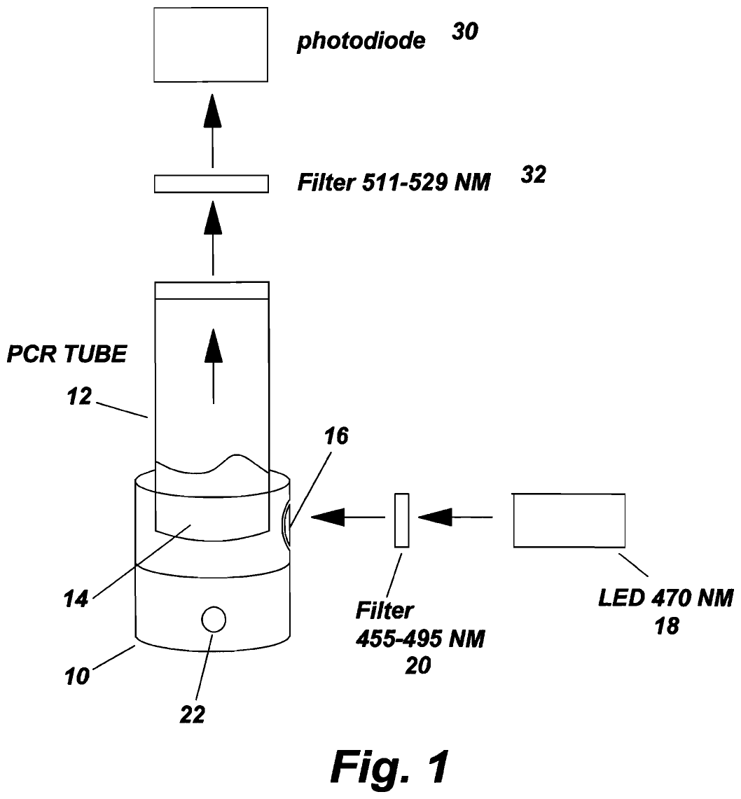

FIG. 2 is an exploded view thereof;

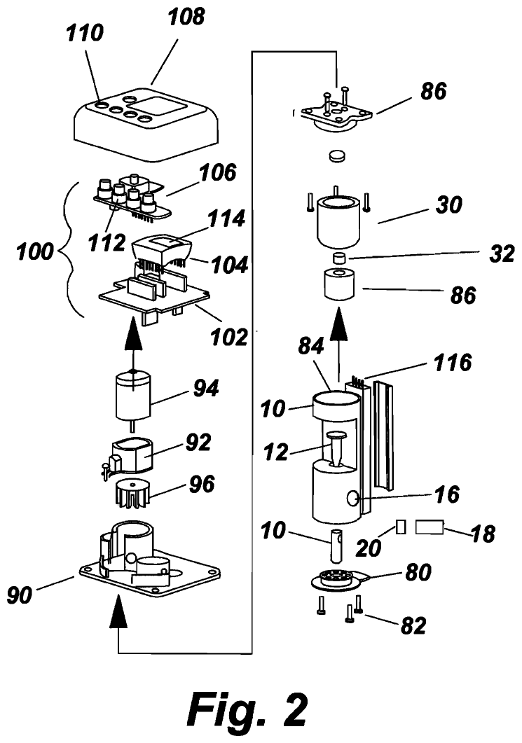

FIG. 3 is a pictorial of accompanying testing devices; and

FIG. 4 is a pictorial of SPIRIT interfaces.

### DETAILED DESCRIPTION OF THE PREFERRED EMBODIMENT

While the present invention is susceptible of embodiment in various forms, there is shown in the drawings and will hereinafter be described a presently preferred and alternative embodiments with the understanding that the present disclosure is to be considered an exemplification of the invention and is not intended to limit the invention to the specific embodiments illustrated.

Disclosed is a PCR device that quantifies target sequence molecules in a sample during a PCR amplification process. The device is compact, lightweight, portable, and battery-operated. The device can perform real-time PCR for amplifying while monitoring a target nucleic acid sequence and detect, as well as quantify, an infection of pathogens.

The PCR device includes a casing, circuit board, and fixtures for a light path that do not depend on complex tooling. A casing for the overall device houses a heating element holder, a fan and cooling system, as well as a structure for fluorescence reading. The device is designed to use a typical PCR tube that has several key features, including a complete seal for inhibiting evaporation of the sample and high optical transparency for the excitation and emission wavelengths. The mechanical parts of the device are designed to be as compact as possible.

Referring to the Figures in general, a heating element 10 is a piece of aluminum constructed and arranged to receive a PCR tube 12. A portion of the PCR tube 12 contains a reaction solution 14 to be inserted into the PCR tube 12. The heating element 10 includes an opening 16 for receipt of a 470 NM LED light 18 that is filtered by a lens 20 having a range of between 455-495 NM. Light from the LED 18 is used to excite the solution 14 placed in the PCR tube 12. A thermistor 22 is used to measure the temperature of the heating element. The heating element consists of an aluminum rod, a polyimide film, and a coil of NiCr wire 116 which is powered by a lithium battery, not shown.

To cool the heating element 10, a centrifugal fan 92 is controlled by a microcontroller 104 using a relay. The fan motor 94 drives an impeller 96 to draw in air at ambient temperature to lower the temperature of the heating block and the sample.

Joule heating from the NiCr wire 116 is applied to the polyimide tape as thermal power. The thermal power applied is determined by measuring the power dissipated by the NiCr wire 116 during heating cycles. In the heating simulations, a fan-less operation is assumed, while the cooling simulations are performed with a higher convection coefficient to model a fan.

The optical components provide a real-time PCR system, as they provide the means of determining the concentration of amplified target DNA. A photodiode 30 and emission filter 32 are located coaxially to the PCR tube 12. The LED 18 emits blue light through the blue excitation filter 20 and travels through the opening 16 in the side of the aluminum heating block 10 to illuminate the sample solution 14 located in the PCR tube 12. The intercalating dyes in the sample emit green light when exposed to this blue excitation light, in intensity proportional to the concentration of the target DNA. This green fluorescence light travels up through the PCR tube 12 and through the emission filter 32 for detection by the photodiode sensor 30. The photodiode sensor 30 produces a voltage corresponding to the fluorescence intensity emitted by the sample. This voltage is then amplified and read by a microcontroller for use in quantifying the amplification of DNA produced by PCR. This optical design adapts a simple separation of the emission and excitation light paths to reduce the direct injection of excitation wavelength into the photodiode.

The PCR tube 12 fits within the heating block element 10, the heating block having a bottom vent 80 that is attached by use of fasteners 82. The heating block 10 has an upper aperture 84 for receipt of the photodiode 30 and emission filter 32. The emission filter 32 is placed within the holder 86. The photodiode 30 and emission filter 32 are attached to the upper bracket 86 for securement as a unit to the heating block 10.

The heating block 10 is coupled to an assembly which further supports a fan housing 92 for joining a fan motor 94 to fan blades 96. A control assembly 100 consists of a circuit board 102 for receipt of a microcontroller 104 and a control board 106. A housing cover 108 has apertures 110 for receipt of preprogrammed controller buttons 112; the microcontroller having a screen 114 for operational display of the preprogrammed microcontroller 104.

In a preferred embodiment, a 1.75 mm opaque black ABS filament is used. Black filament is used so that the case and optical components are opaque to avoid interference with the fluorescent measurements. To integrate the heating, cooling, and optical systems, the circuit board 102 is made to connect all elements together.

The electronic control system is based on a microcontroller 104 with a built in OLED screen 114. The microcontroller 104 is used to control relays that operate the fan motor 94 and the NiCr wire 116, and is also responsible for turning on the blue excitation LED and obtaining a reading from the photodiode 30. Additionally, the microcontroller 104 receives input from the user via a set of buttons 112, and delivers output to the user by way of the built in OLED screen 114. The software that controls the operation of the device and runs on the microcontroller is responsible for reading the buttons, controlling the heater block 10 and fan motor 94, enabling the LED, measuring the fluorescent reading, and displaying information on the screen. The temperature control works using a thermistor 22 mounted on the aluminum heating block 10. This thermistor 22 is connected in series with a 10 kΩ resistor and creates a voltage divider. The result is a voltage that is proportional to the temperature of the aluminum block. This voltage is read by the microcontroller 104 and converted to degrees Celsius. When in a thermocycling mode, the microcontroller 104 enables the heater block 10. Once the temperature reaches a high threshold, the fan motor 94 is enabled and the heater block 10 is disabled. The fan blows ambient temperature air over the aluminum block and cools until the low threshold is reached. The fan motor 94 is then disabled and the heater block 10 enabled for short pulses until the temperature inside of the tube finally reaches the low threshold. At this point, the LED is enabled and five readings are taken from the photodiode sensor 30. The median of these five samples is recorded to avoid any errors caused by noise on the reading.

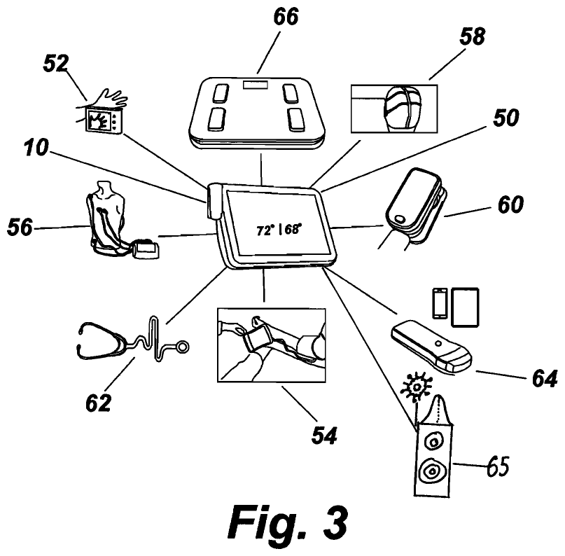

Referring to FIG. 3, the PCR tube 12 can be coupled to an Alexis unit for use in combination with other medical devices to get information of body temperature 52, blood pressure 54, EKG 56, EEG 58, blood oxygen saturation level 60, blood flow and stethoscope sound 62, ultrasound imaging 64, high volume air sampler for nano particles 65, body weight and body fat 66 and so forth, to obtain the user's physical exam data. Addition of Bluetooth and other technologies can be made to increase expandability and connection to other devices.

In another embodiment, the PCR tube 12 can be coupled to a high volume air sampler for nanoparticles 65. This can be an independent stand alone instrument, or with the PCR incorporated as a component of the instrument. In this embodiment, the device is used to collect/acquire samples of environmental pathogens (virus, bacteria, fungus) or other air pollutants of nanoparticle size and notate the location where the sample was collected. Once collected, the samples are then analyzed using the PCR device to identify the type of pathogen and concentration level.

Combination of the PCR tube and other instruments mentioned above with a network communication device (TOTUS) can be utilized to send the physical exam and PCR information to servers, and/or physicians, pharmacists and other medical professionals. TOTUS should have user's voice recognition to control all the functions by voice command. TOTUS should have face recognition to recognize the user who is accessing the device. TOTUS should have fingertip recognition to control all the functions and recognize the sign language to communicate to the user.

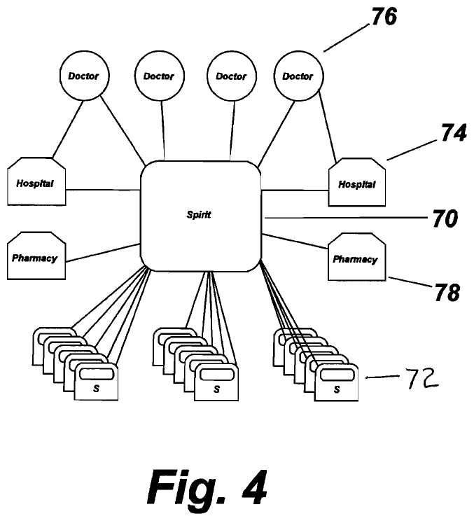

Referring to FIG. 4, the server 70 (SPIRIT, SuPer IntegRated Information Technology) receiving the information from TOTUS has a capability of AI to assist diagnosis. The function will be supported by searching an accumulated medical information database 72 with the physical exam numerical and image data. It will reference the user's individual personal medical record information as well. SPIRIT guides the user whether to go to the hospital 74 or stay at home. SPIRIT picks the best matched physician 76 in the area for the user based on the data collected by TOTUS, the medical information database, and the user's medical record to make an appointment. SPIRIT makes arrangements with the pharmacy 78, forwards the prescription with the personal medical record, including medication history, drug allergy and drug interaction to other medications information which has been prescribed to the user, and other information related to medication.

SPIRIT makes arrangements with the pharmacy to send the prescribed medication to the user via mail, UBER and other delivery systems, or makes arrangements for the prescribed medication to be ready for pick up by the user. SPIRIT will follow up with the user to determine whether the prescribed medication has been properly taken, efficacy of the medication, and the recovery by information from TOTUS. If it is necessary, SPIRIT will ask the user to take medication and/or conduct further testing through TOTUS. All the follow up data will also be stored in SPIRIT for future use.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification.

The term “coupled” is defined as connected, although not necessarily directly, and not necessarily mechanically. The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

One skilled in the art will readily appreciate that the present invention is well adapted to carry out the objects and obtain the ends and advantages mentioned, as well as those inherent therein. Any compounds, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A polymerase chain reaction device comprising:
a heating element;
a casing coupled to said heating element, said casing containing a reaction solution;
a 470 NM LED filtered by a lens having a range between 455-495 NM to produce a light to excite said reaction solution;
a photodiode sensor filtered by a lens in the range of 511-529 NM for detection of light passed through said excited reaction solution, said photodiode sensor producing a voltage corresponding to a fluorescence intensity emitted by said excited reaction solution;
an amplifier coupled to said photodiode sensor to amplify the voltage;
a microcontroller for use in quantifying the amplification of voltage, wherein an intensity of voltage is proportional to the fluorescence intensity concentration of target DNA in said reaction solution;
a thermistor used to measure the temperature of said heating element; and
a centrifugal fan positioned in said casing to draw air past said heating element, said microcontroller coupled to said centrifugal fan to control the temperature of said heating element and said reaction solution, wherein said microcontroller includes a display of the fluorescence intensity of the reactive solution.

2. The polymerase chain reaction device according to claim 1 wherein said heating element consists of an aluminum rod, a polyimide film, and a coil of NiCr wire.

3. The polymerase chain reaction device according to claim 2 wherein said heating element is heated by a lithium battery.

4. The polymerase chain reaction device according to claim 2 wherein Joule heating from said coil of NiCr wire is applied to a polyimide tape as thermal power, and the thermal power applied is determined by measuring the power dissipated by the NiCr wire during heating cycles.

5. The polymerase chain reaction device according to claim 1 wherein said microcontroller is coupled to an Alexis unit for use in combination with other medical devices to obtain information selected from the group consisting of body temperature, blood pressure, EKG, EEG, blood oxygen saturation level, blood flow and stethoscope sound, ultrasound imaging, body weight, and body fat.

6. The polymerase chain reaction device according to claim 1 wherein said microcontroller is coupled to a high volume air sampler for detection of nano-particles.

7. The polymerase chain reaction device according to claim wherein the nano-particles are selected from the group consisting of environmental pathogens, namely virus, bacteria, fungus, or other air pollutants of nano-particle size.

8. The polymerase chain reaction device according to claim 1 wherein said microcontroller is coupled to a SuPer IntegRated Information Technology software receiving information from TOTUS to assist in diagnosis and determining if a user should seek medical assistance.

9. The polymerase chain reaction device according to claim 8 wherein said SuPer IntegRated Information Technology software picks the best matched physician for the user based on the data collected by TOTUS.
